(19) United States
(12) Patent Application Publication
(10) Pub. No.: US 2016/0235369 A1
(43) Pub. Date: Aug. 18, 2016

(54) MEASURING INSTRUMENT

(71) Applicant: PIONEER CORPORATION, Kanagawa (JP)

(72) Inventors: Kunihiko HORIKAWA, Tokyo (JP); Eisaku KAWANO, Tokyo (JP); Yutaka MATSUI, Tokyo (JP)

(21) Appl. No.: 15/024,629

(22) PCT Filed: Sep. 27, 2013
(86) PCT No.: PCT/JP2013/076313

§ 371 Date: Mar. 24, 2016

(51) Int. Cl. A61B 5/00; A61B 5/024

(52) U.S. Cl. CPC A61B 5/7214; A61B 5/02427; A61B 2562/0238

(57) ABSTRACT

A measuring instrument is provided with: a light emitting device (201) configured to irradiate light; a first light receiving device (211) and a second light receiving device (212) configured to receive return light of the irradiated light, which returns from an object (500) to be measured; and a calculating device (320) configured to calculate information regarding the object to be measured, on the basis of a difference between a sum signal and a difference signal, each of which is calculated from the light received by the first light receiving device and the light received by the second light receiving device. According to the measuring instrument, it is possible to remove unnecessary component, such as a body motion, and to accurately measure information on the object to be measured.

### TECHNICAL FIELD

The present invention relates to a measuring instrument configured to measure various information, such as biological information, for example, on the basis of return light returning from an object to be measured.

### BACKGROUND ART

In this type of measuring instrument, for example, a living body, which is an object to be measure, is irradiated with light emitted from a light emitting element, and biological information, such as pulsation, is measured on the basis of return light detected by a light receiving element. There may be one light receiving element that is used for the measuring instrument; however, two or more light receiving elements can be used to realize more preferable measurement.

For example, in Patent Literature 1, there is proposed a technology in which the pulsation is detected on the basis of a difference signal of two light receiving elements. In Patent Literature 2, there is proposed a technology in which a body motion is detected on the basis of a difference signal of two light receiving elements. In Patent Literature 3, there is proposed a technology in which two light receiving elements are set to have an equal distance from one light emitting element, in order to prevent superposition of an offset component, which is not required for a tracking signal.

### CITATION LIST

### Patent Literature

Patent Literature 1: International Publication No. 99/12469

Patent Literature 2: Japanese Patent No. 3789487

Patent Literature 3: Japanese Patent No. 3966434

### SUMMARY OF INVENTION

### Technical Problem

When the biological information is detected, in some cases, the body motion of the living body that is being measured or the like causes a disturbance in a detection signal. Thus, in order to accurately detect the biological information, it is preferable that an influence by the body motion can be removed from the detection signal. In the method of detecting the body motion as described in the Patent Literature 2, however, if the light receiving elements are distant from each other, the light receiving elements have different paths of light that propagates in the living body. As a result, different body motions are detected. For example, if the pulsation is detected in a finger of the living body or the like, different body motions are detected between a fingertip and a base of a finger. As described above, in the conventional technologies including the aforementioned Patent Literatures, the body motion is hardly accurately detected.

In view of the aforementioned technical problems, it is therefore an object of the present invention to provide a measuring instrument that can remove unnecessary components, such as the body motion, and that can accurately measure information on an object to be measured.

### Solution to Problem

The above object of the present invention can be achieved by a measuring instrument comprising: a light emitting device configured to irradiate light; a first light receiving device and a second light receiving device configured to receive return light of the irradiated light, which returns from an object to be measured; and a calculating device configured to calculate information regarding the object to be measured, on the basis of a difference between a sum signal and a difference signal, each of which is calculated from the light received by the first light receiving device and the light received by the second light receiving device.

The above object of the present invention can be achieved by a measuring instrument comprising: a light emitting device configured to irradiate light; a first light receiving device pair and a second light receiving device pair, each of which has two light receiving devices configured to receive return light of the irradiated light, which returns from an object to be measured; a sum signal generating device configured to add an output of one light receiving device and an output of the other light receiving device to make an addition output, in each of the first light receiving device pair and the second light receiving device pair, and configured to add the addition output of the first light receiving device pair and the addition output of the second light receiving device pair to generate a sum signal; a difference signal generating device configured to subtract one of an output of one light receiving device and an output of the other light receiving device from the other to make a subtraction output, in each of the first light receiving device pair and the second light receiving device pair, and configured to add the subtraction output of the first light receiving device pair and the subtraction output of the second light receiving device pair to generate a difference signal; and a calculating device configured to calculate information regarding the object to be measured, on the basis of a difference between the sum signal and the difference signal.

The above object of the present invention can be achieved by a measuring instrument comprising: a light emitting device configured to irradiate light; a first light receiving device, a second light receiving device, a third light receiving device, and a fourth light receiving device, configured to receive return light of the irradiated light, which returns from an object to be measured; a first arithmetic operating device configured to arithmetically operate a first addition output and a first subtraction output, the first addition output being obtained by adding outputs of the first light receiving device and the second light receiving device, which are adjacent to each other in one direction, and the first subtraction output being obtained by subtracting one of the outputs of the first light receiving device and the second light receiving device from the other; a second arithmetic operating device configured to arithmetically operate a second addition output and a second subtraction output, the second addition output being obtained by adding outputs of the third light receiving device and the fourth light receiving device, which are adjacent to each other in the one direction, and the second subtraction output being obtained by subtracting one of the outputs of the third light receiving device and the fourth light receiving device from the other; a third arithmetic operating device configured to arithmetically operate a third addition output and a third subtraction output, the third addition output being obtained by adding outputs of the first light receiving device and the third light receiving device, which are adjacent to each other in another direction that is different from the one direction, and the third subtraction output being obtained by subtracting one of the outputs of the first light receiving device and the third light receiving device from the other; a fourth arithmetic operating device configured to arithmetically operate a fourth addition output and a fourth subtraction output, the fourth addition output being obtained by adding outputs of the second light receiving device and the fourth light receiving device, which are adjacent to each other in the another direction, and the fourth subtraction output being obtained by subtracting one of the outputs of the second light receiving device and the fourth light receiving device from the other; a first sum signal generating device configured to add the first addition output and the second addition output to generate a sum signal regarding the one direction; a first difference signal generating device configured to add the first subtraction output and the second subtraction output to generate a difference signal regarding the one direction; a second sum signal generating device configured to add the third addition output and the fourth addition output to generate a sum signal regarding the another direction; a second difference signal generating device configured to add the third subtraction output and the fourth subtraction output to generate a difference signal regarding the another direction; and a calculating device configured to calculate information regarding the object to be measured, on the basis of a difference between the sum signal and the difference signal, in each of the one direction and the another direction.

The operation and other advantages of the present invention will become more apparent from an embodiment and examples explained below.

### BRIEF DESCRIPTION OF DRAWINGS

FIG. 1 is a schematic block diagram illustrating an entire configuration of a measuring instrument according to a first example.

FIG. 2 is a perspective view illustrating a method of measuring biological information by using the measuring instrument according to the first example.

FIG. 3 is a plan view illustrating a distance relation between a light emitting element and light receiving elements of the measuring instrument according to the first example.

FIG. 4 is a flowchart illustrating an operation of the measuring instrument according to the first example.

FIG. 5 is version 1 of a graph illustrating one example of a sum signal.

FIG. 6 is version 1 of a graph illustrating one example of a difference signal.

FIG. 7 is version 1 of a graph illustrating one example of a differential signal between the sum signal and the difference signal.

FIG. 8 is version 2 of a graph illustrating one example of the sum signal.

FIG. 9 is version 2 of a graph illustrating one example of the difference signal.

FIG. 10 is version 2 of a graph illustrating one example of the differential signal between the sum signal and the difference signal.

FIG. 11 is a plan view illustrating a distance relation between a light emitting element and light receiving elements of a measuring instrument according to a second example.

FIG. 12 is a plan view illustrating a measuring instrument in a modified example according to the second example.

FIG. 13 is a flowchart illustrating an operation of the measuring instrument according to the second example.

FIG. 14 is a plan view illustrating a distance relation between light receiving elements of a measuring instrument according to a third example.

FIG. 15 is a flowchart illustrating an operation of the measuring instrument according to the third example.

### DESCRIPTION OF EMBODIMENT

### Embodiment of the Invention

Hereinafter, a measuring instrument according to an embodiment will be explained.

A first measuring instrument according to the embodiment provide with a light emitting device configured to irradiate light; a first light receiving device and a second light receiving device configured to receive return light of the irradiated light, which returns from an object to be measured; and a calculating device configured to calculate information regarding the object to be measured, on the basis of a difference between a sum signal and a difference signal, each of which is calculated from the light received by the first light receiving device and the light received by the second light receiving device.

According to the first measuring instrument in the embodiment, in operation thereof, a living body or the like, which is the object to be measured, is irradiated with light emitted from the light emitting device, which includes, for example, a light emitting diode (LED) or the like. The light irradiated from the light emitting diode is scattered in or transmitted through the object to be measured, and is detected as the return light on both the first light receiving device and the second light receiving device. Each of the first light receiving device and the second light receiving device includes a photodiode or the like, and outputs a detection signal according to the detected return light.

If the detection signals are respectively outputted from the first light receiving device and the second light receiving device, the sum signal and the difference signal of the detection signals are calculated on the calculating device, which includes, for example, an arithmetic circuit. In other words, the sum signal obtained by adding the detection signal detected by the first light receiving device and the detection signal detected by the second light receiving device, and the difference signal, which is a difference between the detection signal detected by the first light receiving device and the detection signal detected by the second light receiving device, are calculated.

Then, the difference between the sum signal and the difference signal is calculated on the calculating device. Then, the information regarding the object to be measured is calculated on the basis of the difference between the sum signal and the difference signal. An example of the information to be calculated can be pulsation in the living body or the like.

Here, in particular, the detection signals respectively obtained from the first light receiving device and the second light receiving device include the same degree of one information regarding the object to be measured (e.g. information that does not vary due to a slight difference in position, such as pulsation components of the living body), whereas the detection signals also include another information regarding the object to be measured (e.g. information that varies due to a slight difference in position, such as body motion components of the living body) in different states. Thus, in the difference signal, which is the difference between the detection signals, the one information regarding the object to be measured is canceled, and only the another information regarding the object to be measured remains. Therefore, only the another information can be extracted from a plurality of types of information obtained from the object to be measured.

Moreover, the another information included in the sum signal and the another information included in the difference signal are canceled out by subtracting the difference signal from the sum signal of the detection signals. As a result, only the one information can be extracted from the plurality of types of information obtained from the object to be measured.

As explained above, according to the measuring instrument in the embodiment, only particular information can be extracted from various types of information included in the detection signals. Specifically, for example, regarding the detection signals obtained from the living body, which is the object to be measured, only the pulsation component or only the body motion component can be extracted. It is therefore possible to remove unnecessary components and to accurately measure information on the object to be measured.

In an aspect of the first measuring instrument according to the embodiment, wherein a distance between a light emitting point of the light emitting device and the first light receiving device is equal to a distance between the light emitting point of the light emitting device and the second light receiving device.

According to this aspect, since a light receiving condition of the first light receiving device and a light receiving condition of the second light receiving device can be set to be similar, particular information can be more accurately extracted from the respective detection signals of the light receiving devices.

The term “equal” in this aspect is a broad concept, including not only a case where the two values completely match, but also a case where the two values are close enough to sufficiently obtain the aforementioned effect.

A second measuring instrument according to the embodiment provide with a light emitting device configured to irradiate light; a first light receiving device pair and a second light receiving device pair, each of which has two light receiving devices configured to receive return light of the irradiated light, which returns from an object to be measured; a sum signal generating device configured to add an output of one light receiving device and an output of the other light receiving device to make an addition output, in each of the first light receiving device pair and the second light receiving device pair, and configured to add the addition output of the first light receiving device pair and the addition output of the second light receiving device pair to generate a sum signal; a difference signal generating device configured to subtract one of an output of one light receiving device and an output of the other light receiving device from the other to make a subtraction output, in each of the first light receiving device pair and the second light receiving device pair, and configured to add the subtraction output of the first light receiving device pair and the subtraction output of the second light receiving device pair to generate a difference signal; and a calculating device configured to calculate information regarding the object to be measured, on the basis of a difference between the sum signal and the difference signal.

According to the second measuring instrument in the embodiment, in operation thereof, a living body or the like, which is the object to be measured, is irradiated with light emitted from the light emitting device, which includes, for example, a LED or the like. The light irradiated from the light emitting diode is scattered in or transmitted through the object to be measured, and is detected as the return light on both the first light receiving device pair and the second light receiving device pair. Each of the first light receiving device pair and the second light receiving device pair is provided with one light receiving device and the other light receiving device. Each of the one light receiving device and the other light receiving device includes a photodiode or the like, and outputs a detection signal according to the detected return light.

If the detection signals are outputted from the first light receiving device pair and the second light receiving device pair, the output of the one light receiving device and the output of the other light receiving device in each of the first light receiving device pair and the second light receiving device pair are added to make the addition output on the sum signal generating device. In other words, the output of the one light receiving device and the output of the other light receiving device in the first light receiving device pair are added to make the addition output of the first light receiving device pair, and the output of the one light receiving device and the output of the other light receiving device in the second light receiving device pair are added to make the addition output of the second light receiving device pair. Then, on the sum signal generating device, the addition output of the first light receiving device pair and the addition output of the second light receiving device pair are added to generate the sum signal. In other words, the sum signal is a signal obtained by adding the detection signals of the four light receiving devices included in the first light receiving device pair and the second light receiving device pair.

On the other hand, one of the output of one light receiving device and the output of the other light receiving device in each of the first light receiving device pair and the second light receiving device pair is subtracted from the other to make the subtraction output on the difference signal generating device. In other words, one of the output of one light receiving device and the output of the other light receiving device in the first light receiving device pair is subtracted from the other to make the subtraction output of the first light receiving device pair, and one of the output of one light receiving device and the output of the other light receiving device in the second light receiving device pair is subtracted from the other to make the subtraction output of the second light receiving device pair. Then, on the difference signal generating device, the subtraction output of the first light receiving device pair and the subtraction output of the second light receiving device pair are added to generate the difference signal. In other words, the difference signal is a signal obtained by adding the difference signal of the two light receiving devices included in the first light receiving device pair and the difference signal of the two light receiving devices included in the second light receiving device pair.

If the sum signal and the difference signal are calculated, the difference between the sum signal and the difference signal is calculated on the calculating device. Then, the information regarding the object to be measured is calculated on the basis of the difference between the sum signal and the difference signal. An example of the information to be calculated can be pulsation in the living body or the like.

Here, in particular, the detection signals respectively obtained from the first light receiving device pair and the second light receiving device pair include the same degree of one information regarding the object to be measured (e.g. information that does not vary due to a slight difference in position, such as pulsation components of the living body), whereas the detection signals also include another information regarding the object to be measured (e.g. information that varies due to a slight difference in position, such as body motion components of the living body) in different states. Thus, in the difference signal obtained by adding the subtraction outputs, each of which is the difference between the detection signals, the one information regarding the object to be measured is canceled, and only the another information regarding the object to be measured remains. Therefore, only the another information can be extracted from a plurality of types of information obtained from the object to be measured.

Moreover, the another information included in the sum signal and the another information included in the difference signal are canceled out by subtracting the difference signal from the sum signal of the detection signals. As a result, only the one information can be extracted from the plurality of types of information obtained from the object to be measured.

As explained above, according to the measuring instrument in the embodiment, only particular information can be extracted from various types of information included in the detection signals. Specifically, for example, regarding the detection signals obtained from the living body, which is the object to be measured, only the pulsation component or only the body motion component can be extracted. It is therefore possible to remove the unnecessary components and to accurately measure the information on the object to be measured.

In an aspect of the second measuring instrument according to the embodiment, wherein a distance between a light emitting point of the light emitting device and one light receiving device of the first light receiving device pair is equal to a distance between the light emitting point of the light emitting device and the other light receiving device of the first light receiving device pair, and a distance between the light emitting point of the light emitting device and one light receiving device of the second light receiving device pair is equal to a distance between the light emitting point of the light emitting device and the other light receiving device of the second light receiving device pair.

According to this aspect, a light receiving condition of the one light receiving device in the first light receiving device pair and a light receiving condition of the other light receiving device in the first light receiving device pair can be set to be similar, and a light receiving condition of the one light receiving device in the second light receiving device pair and a light receiving condition of the other light receiving device in the second light receiving device pair can be set to be similar. Thus, particular information can be more accurately extracted from the respective detection signals of the light receiving devices.

The term “equal” in this aspect is a broad concept, including not only the case where the two values completely match, but also the case where the two values are close enough to sufficiently obtain the aforementioned effect.

A third measuring instrument according to the embodiment provide with a light emitting device configured to irradiate light; a first light receiving device, a second light receiving device, a third light receiving device, and a fourth light receiving device, configured to receive return light of the irradiated light, which returns from an object to be measured; a first arithmetic operating device configured to arithmetically operate a first addition output and a first subtraction output, the first addition output being obtained by adding outputs of the first light receiving device and the second light receiving device, which are adjacent to each other in one direction, and the first subtraction output being obtained by subtracting one of the outputs of the first light receiving device and the second light receiving device from the other; a second arithmetic operating device configured to arithmetically operate a second addition output and a second subtraction output, the second addition output being obtained by adding outputs of the third light receiving device and the fourth light receiving device, which are adjacent to each other in the one direction, and the second subtraction output being obtained by subtracting one of the outputs of the third light receiving device and the fourth light receiving device from the other; a third arithmetic operating device configured to arithmetically operate a third addition output and a third subtraction output, the third addition output being obtained by adding outputs of the first light receiving device and the third light receiving device, which are adjacent to each other in another direction that is different from the one direction, and the third subtraction output being obtained by subtracting one of the outputs of the first light receiving device and the third light receiving device from the other; a fourth arithmetic operating device configured to arithmetically operate a fourth addition output and a fourth subtraction output, the fourth addition output being obtained by adding outputs of the second light receiving device and the fourth light receiving device, which are adjacent to each other in the another direction, and the fourth subtraction output being obtained by subtracting one of the outputs of the second light receiving device and the fourth light receiving device from the other; a first sum signal generating device configured to add the first addition output and the second addition output to generate a sum signal regarding the one direction; a first difference signal generating device configured to add the first subtraction output and the second subtraction output to generate a difference signal regarding the one direction; a second sum signal generating device configured to add the third addition output and the fourth addition output to generate a sum signal regarding the another direction; a second difference signal generating device configured to add the third subtraction output and the fourth subtraction output to generate a difference signal regarding the another direction; and a calculating device configured to calculate information regarding the object to be measured, on the basis of a difference between the sum signal and the difference signal, in each of the one direction and the another direction.

According to the third measuring instrument in the embodiment, in operation thereof, a living body or the like, which is the object to be measured, is irradiated with light emitted from the light emitting device, which includes, for example, a LED or the like. The light irradiated from the light emitting diode is scattered in or transmitted through the object to be measured, and is detected as the return light on the first light receiving device, the second light receiving device, the third light receiving device, and the fourth light receiving device. Each of the first to fourth light receiving devices includes a photodiode or the like, and outputs a detection signal according to the detected return light.

If the detection signals are outputted from the first to fourth light receiving devices, the first addition output and the first subtraction output are arithmetically operated on the first arithmetic operating device, wherein the first addition output is obtained by adding the outputs of the first light receiving device and the second light receiving device, which are adjacent to each other in one direction, and the first subtraction output is obtained by subtracting one of the outputs of the first light receiving device and the second light receiving device from the other. On the second arithmetic operating device, the second addition output and the second subtraction output are arithmetically operated, wherein the second addition output is obtained by adding the outputs of the third light receiving device and the fourth light receiving device, which are adjacent to each other in the one direction, and the second subtraction output is obtained by subtracting one of the outputs of the third light receiving device and the fourth light receiving device from the other. On the third arithmetic operating device, the third addition output and the third subtraction output are arithmetically operated, wherein the third addition output is obtained by adding outputs of the first light receiving device and the third light receiving device, which are adjacent to each other in another direction that is different from the one direction, and the third subtraction output is obtained by subtracting one of the outputs of the first light receiving device and the third light receiving device from the other. On the fourth arithmetic operating device, the fourth addition output and the fourth subtraction output are arithmetically operated, wherein the fourth addition output is obtained by adding outputs of the second light receiving device and the fourth light receiving device, which are adjacent to each other in the another direction, and the fourth subtraction output is obtained by subtracting one of the outputs of the second light receiving device and the fourth light receiving device from the other.

The first addition output and the second addition output are added on the first sum signal generating device to generate the sum signal regarding the one direction. Moreover, the first subtraction output and the second subtraction output are added on the first difference signal generating device to generate the difference signal regarding the one direction. In the same manner, the third addition output and the fourth addition output are added on the second sum signal generating device to generate the sum signal regarding the another direction. Moreover, the third subtraction output and the fourth subtraction output are added on the second difference signal generating device to generate the difference signal regarding the another direction. In other words, the sum signal and the difference signal are generated regarding each of the one direction and the another direction.

If the sum signal and the difference signal are calculated, the difference between the sum signal and the difference signal regarding each direction is calculated on the calculating device. Specifically, the difference between the sum signal regarding the one direction and the difference signal regarding the one direction is calculated, and the difference between the sum signal regarding the another direction and the difference signal regarding the another direction is calculated. In other words, the difference between the sum signal and the difference signal is generated regarding each of the one direction and the another direction.

If the difference between the sum signal and the difference signal is calculated, the information regarding the object to be measured is calculated on the basis of the calculated difference regarding each direction. For example, the information regarding the object to be measured is calculated on the basis of the difference regarding the one direction, and the information regarding the object to be measured is calculated on the basis of the difference regarding the another direction. Performing a predetermined arithmetic operation (e.g. an average value calculation, etc.) by using the information calculated on the basis of the difference regarding the one direction and the information calculated on the basis of the difference regarding the another direction also makes it possible to calculate the information based on both the difference regarding the one direction and the difference regarding the another direction. An example of the information regarding the living body to be calculated here can be pulsation in the living body or the like.

Here, in particular, the detection signals respectively obtained from the first to fourth light receiving devices include the same degree of one information regarding the object to be measured (e.g. information that does not vary due to a slight difference in position, such as pulsation components of the living body), whereas the detection signals also include another information regarding the object to be measured (e.g. information that varies due to a slight difference in position, such as body motion components of the living body) in different states. Thus, in the difference signal obtained by adding the differences of the respective detection signals, the one information regarding the object to be measured is canceled, and only the another information regarding the object to be measured remains. Therefore, only the another information can be extracted from a plurality of types of information obtained from the object to be measured.

Moreover, the another information included in the sum signal and the another information included in the difference signal are canceled out by subtracting the difference signal from the sum signal of the detection signals. As a result, only the one information can be extracted from the plurality of types of information obtained from the object to be measured.

Moreover, particularly in the embodiment, the difference between the sum signal and the difference signal is calculated regarding each of the one direction and the another direction, the information regarding the living body can be calculated, more accurately, than in a case where the difference between the sum signal and the difference signal is calculated regarding only the one direction.

As explained above, according to the measuring instrument in the embodiment, only particular information can be extracted from various types of information included in the detection signals. Specifically, for example, regarding the detection signals obtained from the living body, which is the object to be measured, only the pulsation component or only the body motion component can be extracted. It is therefore possible to remove the unnecessary components and to accurately measure the information on the object to be measured.

In an aspect of the third measuring instrument according to the embodiment, wherein the first light receiving device, the second light receiving device, the third light receiving device, and the fourth light receiving device are positioned to have an equal adjacent distance in a planar manner.

According to this aspect, the first to fourth light receiving devices are positioned such that a distance between the first light receiving device and the second light receiving device, which are adjacent to each other in one direction, a distance between the third light receiving device and the fourth light receiving device, which are adjacent to each other in the one direction, a distance between the first light receiving device and the third light receiving device, which are adjacent to each other in another direction, and a distance between the second light receiving device and the fourth light receiving device, which are adjacent to each other in the another direction are equal to each other.

As a result, the first addition output and the first subtraction output, which are respectively obtained by the addition and the subtraction of the outputs of the first light receiving device and the second light receiving device, which are adjacent to each other in the one direction, the second addition output and the second subtraction output, which are respectively obtained by the addition and the subtraction of the outputs of the third light receiving device and the fourth light receiving device, which are adjacent to each other in the one direction, the third addition output and the third subtraction output, which are respectively obtained by the addition and the subtraction of the outputs of the first light receiving device and the third light receiving device, which are adjacent to each other in the another direction, and the fourth addition output and the fourth subtraction output, which are respectively obtained by the addition and the subtraction of the outputs of the second light receiving device and the fourth light receiving device, which are adjacent to each other in the another direction, are calculated in the same condition. In other words, the addition outputs and the subtraction outputs are calculated as parameters regarding the two light receiving elements arranged at predetermined intervals.

Thus, the sum signal and the difference signal respectively obtained by adding the addition outputs and the subtraction outputs can be set appropriate. It is therefore possible to accurately measure information on the object to be measured.

In another aspect of the third measuring instrument according to the embodiment, wherein the first light receiving device, the second light receiving device, the third light receiving device, and the fourth light receiving device have an equal distance from a light emitting point of the light emitting device.

According to this aspect, the respective light receiving conditions of the first to fourth light receiving devices can be set to be similar. Therefore, particular information can be more accurately extracted from the respective detection signals of the light receiving devices.

The term “equal” in this aspect is a broad concept, including not only the case where the two values completely match, but also the case where the two values are close enough to sufficiently obtain the aforementioned effect.

In another aspect of the measuring instrument according to the embodiment, wherein the calculating device calculates the information regarding the object to be measured, on the basis of a difference between a frequency component of the sum signal and a frequency component of the difference signal.

According to this aspect, a frequency analysis is performed on the sum signal and the difference signal, and the frequency component, which is an analysis result, is used to calculate the information regarding the object to be measured. In this manner, particular information can be detected by using a peak of the frequency component, and thus, more preferable measurement can be realized.

In the aspect in which the frequency component is used, as described above, wherein the measuring instrument further comprises a normalizing device configured to divide the frequency component of the sum signal and the frequency component of the difference signal by respective maximum values, to be normalized, and the calculating device calculates the information regarding the object to be measured, on the basis of a difference frequency component, which is a difference between the normalized frequency component of the sum signal and the normalized frequency component of the difference signal.

In this case, the frequency component of the sum signal is divided by the maximum value of the frequency component of the sum signal and is normalized. In the same manner, the frequency component of the difference signal is divided by the maximum value of the frequency component of the difference signal and is normalized. Thus, the peak of the frequency component can be easily detected, and thus, more preferable measurement can be realized.

In the aspect in which the normalized frequency component is used, as described above, wherein the calculating device estimates a frequency that indicates a maximum amplitude in the difference frequency component, to be a pulsation period of the object to be measured.

In this case, the sum signal includes the pulsation component and the body motion component of the living body, which is the object to be measured. On the other hand, the difference signal includes only the body motion component because the pulsation component is canceled. Thus, if the difference frequency component, which is the difference in the frequency component between the sum signal and the difference signal, is calculated, only the body motion components included in the sum signal and the difference signal are canceled out, and only the pulsation component remains. Thus, the frequency that indicates the maximum amplitude in the difference frequency component can be estimated to be the pulsation period of the living body.

### EXAMPLES

Hereinafter, with reference to the drawings, a measuring instrument according to examples will be explained

### 1: First Example

Firstly, a measuring instrument according to a first example will be explained. Hereinafter, an example in which the measuring instrument is applied to a pulsation measuring apparatus will be explained (the same will apply to the subsequent examples).

<1-1: Configuration of Measuring Instrument>

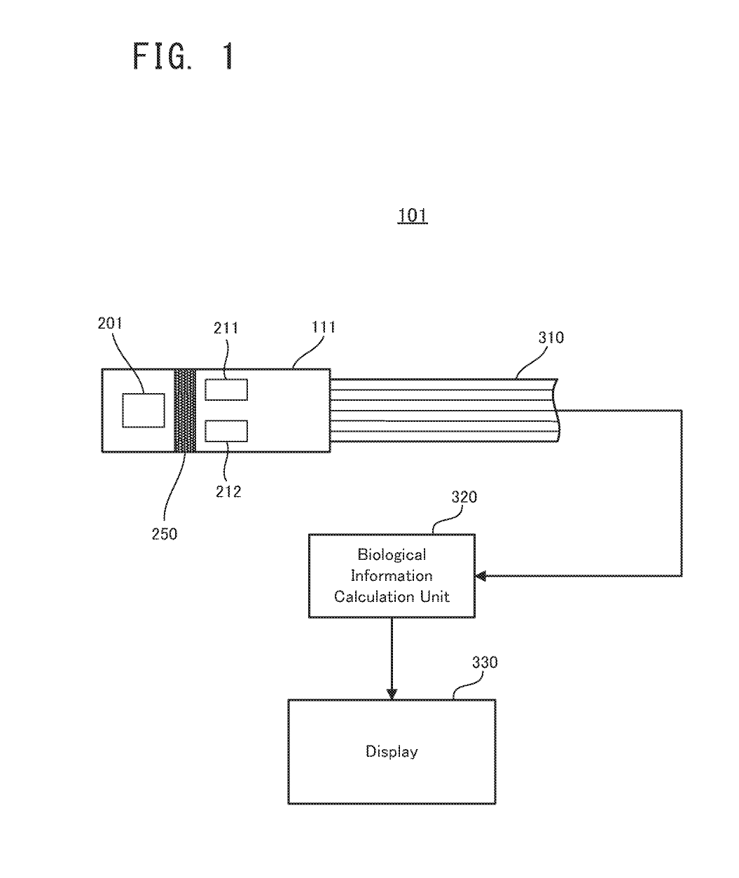

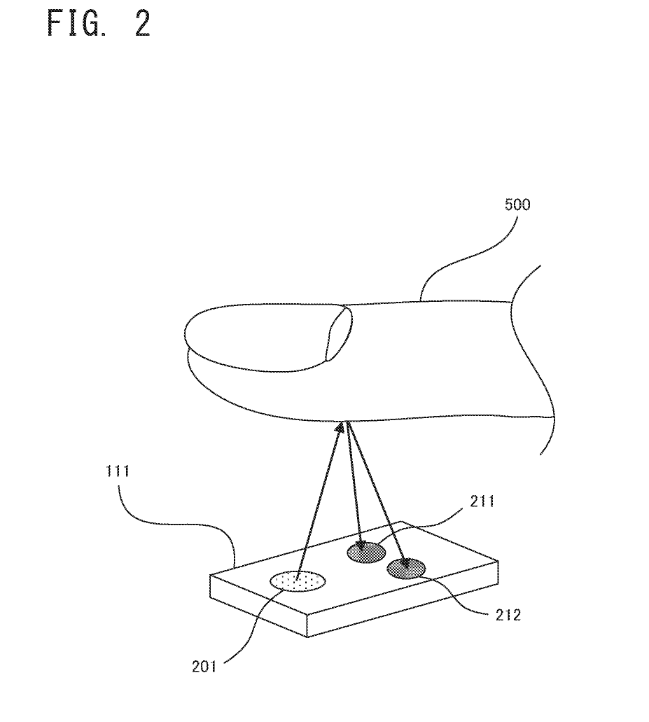

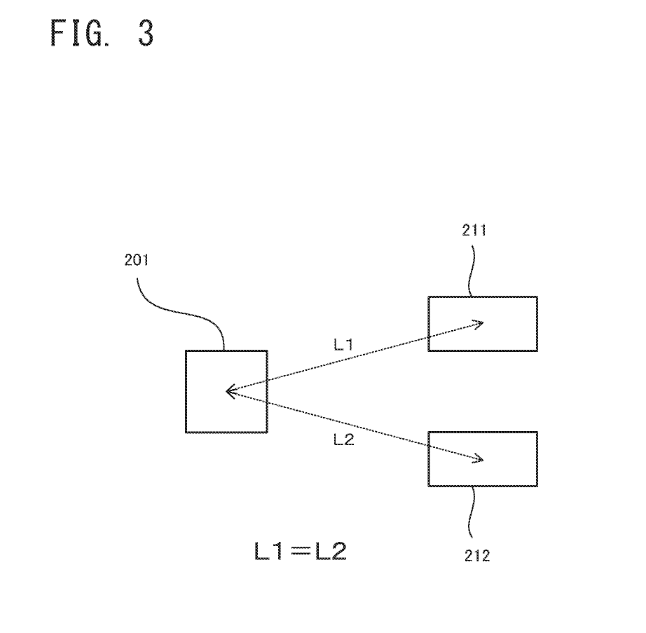

Firstly, a configuration of the measuring instrument according to the first example will be explained with reference to FIG. 1 to FIG. 3. FIG. 1 is a schematic block diagram illustrating an entire configuration of the measuring instrument according to the first example. FIG. 2 is a perspective view illustrating a method of measuring biological information by using the measuring instrument according to the first example. FIG. 3 is a plan view illustrating a distance relation between a light emitting element and light receiving elements of the measuring instrument according to the first example.

In FIG. 1, a measuring instrument 101 according to the example is provided with a probe 111, a wire part 310, a biological information calculation unit 320, and a display 330.

The probe 111 is provided with: a light emitting element 201 configured to emit light to a living body, which is an object to be measured; a first light receiving element 211 and a second light receiving element 212 configured to receive return light returning from the living body; and a shielding plate 250. The light emitting element 201 is one specific example of the “light emitting device”, and includes, for example, one or a plurality of LEDs. The first light receiving element 211 and the second light receiving element 212 are respectively one example of the “first light receiving device” and the “second light receiving device” and are configured as photodiodes having the same structure. The shielding plate 250 is provided to prevent the light irradiated from the light receiving element 201 from being directed to the first light receiving element 211 and the second light receiving element 212 directly (i.e. without being scattered in or transmitted through the living body).

As illustrated in FIG. 2, at the time of measurement, the probe 111 according to the example is mounted on a living body 500 (e.g. a fingertip, an ear lobe, etc.) (In FIG. 2, for convenience of explanation, there is a space between the living body 500 and the probe 111; however, typically, the probe 111 is mounted in such a manner that the living body 500 and the probe 111 are in contact). At the time of measurement, light reflected by the living body 500 out of the light irradiated from the light emitting element 201 is received by the first light receiving element 211 and the second light receiving element 212.

In the example, as described above, a reflective apparatus configured to receive reflected light of the living body 500 is explained; however, the present invention can be also applied to a transmission type apparatus configure to receive transmitted light of the living body 500. In the transmission type apparatus, the first light receiving element 211 and the second light receiving element 212 are disposed opposite the living body 500, as viewed from the light emitting element 201.

As illustrated in FIG. 3, in the probe 111 according to the example, a distance relation between the light emitting element 201 and the first light receiving element 211 or the second light receiving element 212 is clearly defined. Specifically, a distance L1 between a light emitting point of the light emitting element 201 and a light receiving point of the first light receiving element 211 is set to be equal to a distance L2 between the light emitting point of the light emitting element 201 and a light receiving point of the second light receiving element 212. Thus, the return light enters the first light receiving element 211 and the second light receiving element 212 in the same condition.

Back in FIG. 1, the probe 111 is connected to the biological information calculation unit 320 via the wire part 310. Moreover, the biological information calculation unit 320 is connected to the display 330.

The biological information calculation unit 320 is one specific example of the “calculating device”, and calculates a pulsation period of the living body 500 on the basis of detection signals respectively generated by the first light receiving element 211 and the second light receiving element 212 (i.e. signals generates according to intensity of the received light). The biological information calculation unit 320 may calculate biological information other than the pulsation period if the biological information can be calculated from the detection signals. The calculated pulsation period is displayed on the display 330.

The measuring instrument 101 according to the example may be provided with another component, such as an inputting device for controlling the operation of the measuring instrument 101, in addition to the aforementioned components.

<1-2: Operation of Measuring Instrument>

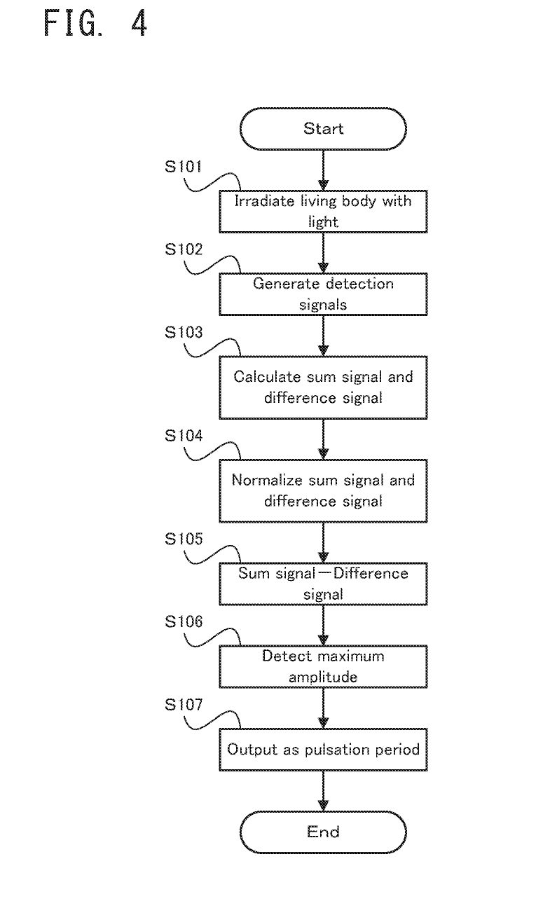

Next, an operation of the measuring instrument according to the first example will be explained with reference to FIG. 4. FIG. 4 is a flowchart illustrating the operation of the measuring instrument according to the first example.

In FIG. 4, in operation of the measuring instrument according to the example, the living body 500 is irradiated with light emitted from the first light emitting element 201 (step S101). The light irradiated from the first light emitting element 201 is reflected by the living body 500, and is received by each of the first light receiving element 211 and the second light receiving element 212. Then, on the first light receiving element 211 and the second light receiving element 212, detection signals are respectively generated according to the intensity of the received light (step S102). In other words, two types of detection signals are separately generated on the first light receiving element 211 and the second light receiving element 212. The detection signals are outputted to the biological information calculation unit 320.

On the biological information calculation unit 320, firstly, a sum signal and a difference signal of the detection signals are calculated (step S103). Specifically, the detection signal of the first light receiving element 211 and the detection signal of the second light receiving element 212 are added, to calculate the sum signal. Moreover, one of the detection signal of the first light receiving element 211 and the detection signal of the second light receiving element 212 is subtracted from the other, to calculate the difference signal.

Then, on the biological information calculation unit 320, the sum signal and the difference signal are normalized (step S104). Specifically, the sum signal is divided by a maximum value of the sum signal, to be normalized. In the same manner, the difference signal is divided by a maximum value of the difference signal, to be normalized. Another normalization method can be also used.

Then, on the biological information calculation unit 320, the normalized difference signal is subtracted from the normalized sum signal (step S105). In other words, a differential signal between the sum signal and the difference signal is calculated. Then, on the biological information calculation unit 320, a frequency with maximum amplitude is detected in the calculated differential signal (step S106), and the detected frequency is outputted as the pulsation period of the living body (step S107).

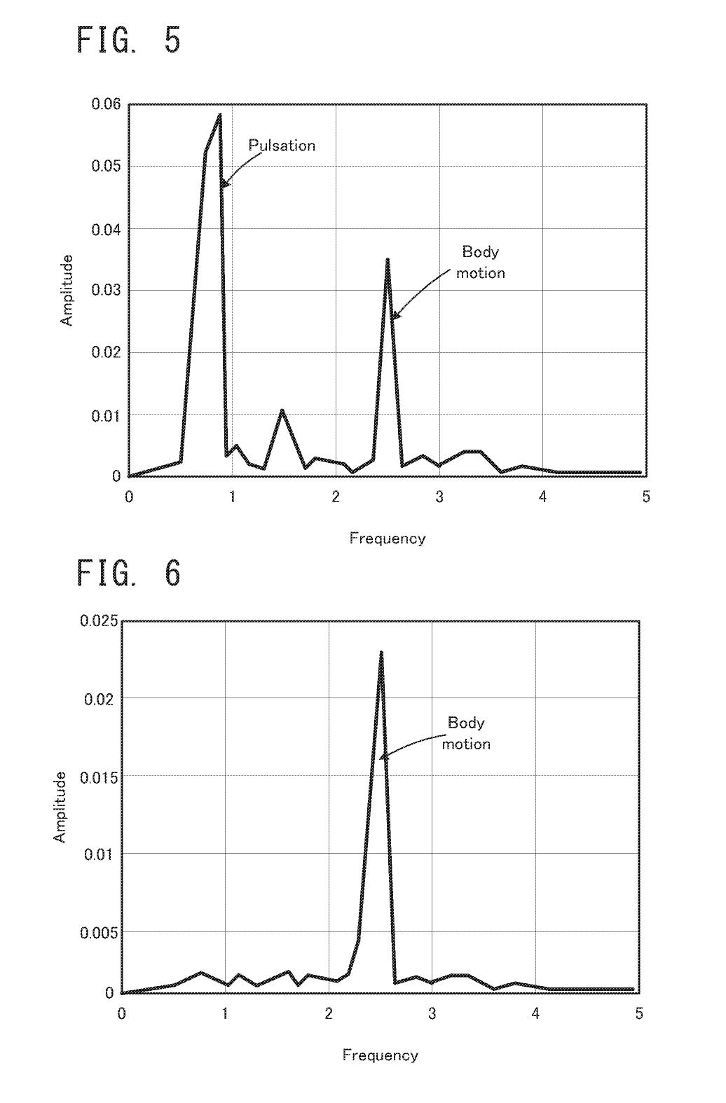

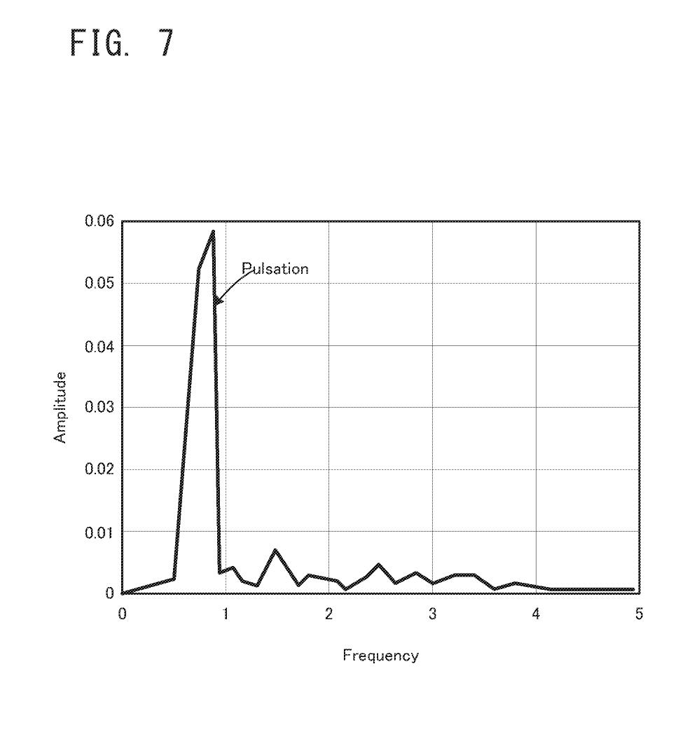

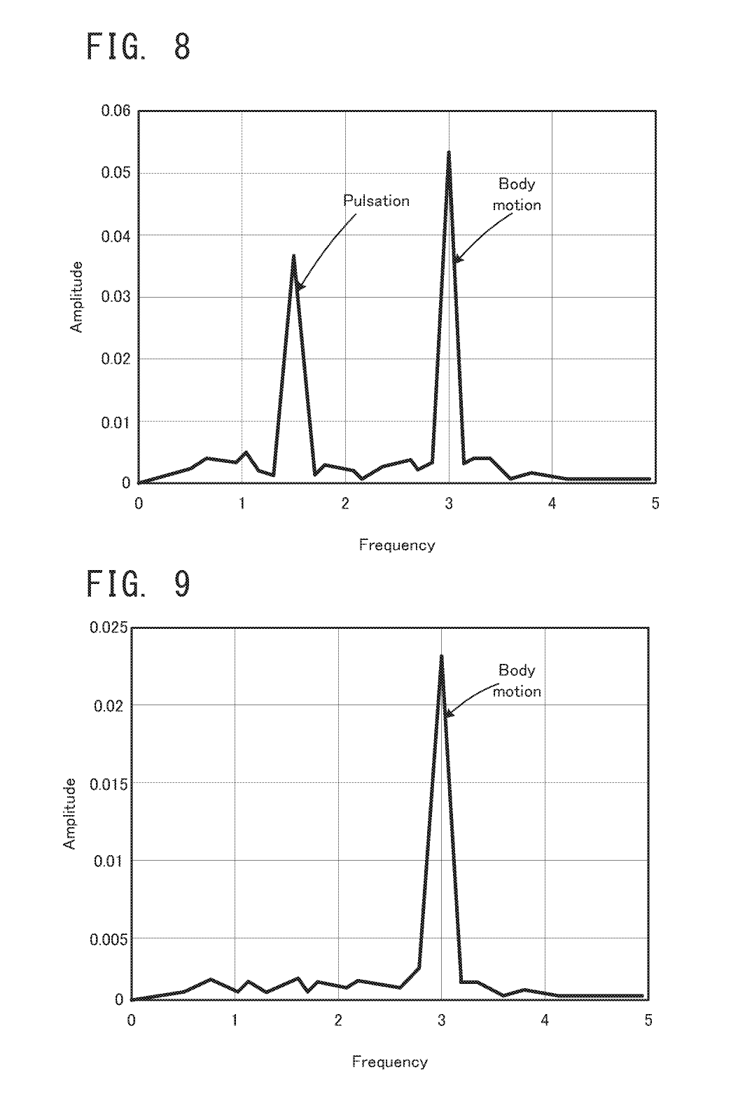

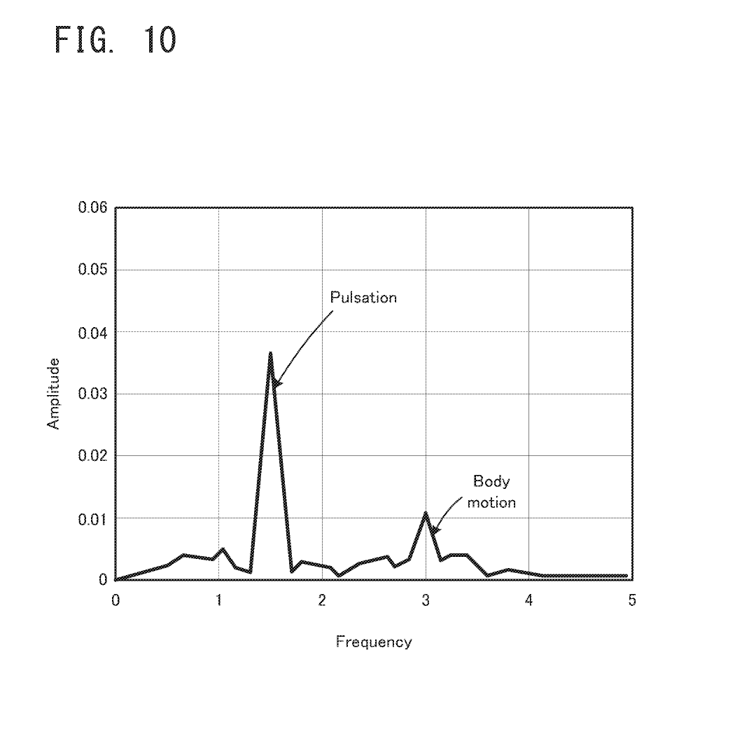

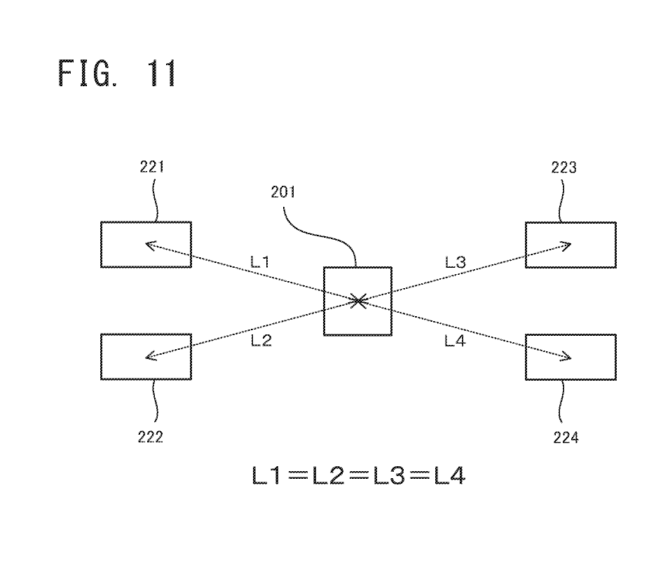

Now, the sum signal and the difference signal calculated from the detection signals, and the differential signal thereof will be specifically explained with reference to FIG. 5 to FIG. 10. FIG. 5 is version 1 of a graph illustrating one example of the sum signal. FIG. 6 is version 1 of a graph illustrating one example of the difference signal. FIG. 7 is version 1 of a graph illustrating one example of the differential signal between the sum signal and the difference signal. FIG. 8 is version 2 of a graph illustrating one example of the sum signal. FIG. 9 is version 2 of a graph illustrating one example of the difference signal. FIG. 10 is version 2 of a graph illustrating one example of the differential signal between the sum signal and the difference signal.]

In FIG. 5, the sum signal is obtained by adding the detection signal of the first light receiving element 211 and the detection signal of the second light receiving element 212. Thus, in the normalized sum signal, the pulsation period of the living body 500 appears as a peak. Here, it is illustrated in such a manner that the peak on the left of the drawing is the pulsation, and the peak on the right is the body motion; however, which peak is the pulsation (i.e. a value to be detected) or not cannot be determined when the sum signal is calculated. Thus, the pulsation cannot be measured by using only the sum signal. Alternatively, even if the measurement can be performed, accuracy is low.

In FIG. 6 the difference signal is obtained by subtracting one of the detection signal of the first light receiving element 211 and the detection signal of the second light receiving element 212 from the other. Here, in particular, as is clear from the sum signal illustrated in FIG. 5, the detection signals respectively obtained from the first light receiving element 211 and the second light receiving element 212 include pulsation components and body motion components of the living body, and have such characteristics that the pulsation component does not vary due to a slight difference in position between the light receiving elements while the body motion component varies due to the slight difference in position between the light receiving elements. Thus, the detection signals respectively obtained from the first light receiving element 211 and the second light receiving element 212 include the same degree of pulsation components and different degrees of body motion components. As a result, in the difference signal, which is a difference between the detection signals, the pulsation component of the living body is canceled, and only the body motion component of the living body remains. Thus, according to the difference signal, only the body motion component can be extracted from a plurality of types of information included in the detection signals.

In FIG. 7, if the difference signal is subtracted from the aforementioned sum signal, the body motion component included in the sum signal and the body motion component included in the difference signal are canceled out. As a result, only the pulsation component of the living body remains in the differential signal. Therefore, the detection of the frequency with the maximum amplitude in the differential signal makes it possible to measure the pulsation period of the living body 500.

In waveforms illustrated in FIG. 5 to FIG. 7, the pulsation period can be detected from the maximum amplitude in the sum signal even without calculating the difference signal and the differential signal; however, the maximum amplitude in the sum signal is not the pulsation component in some cases.

In the sum signal illustrated in FIG. 8, a peak of the pulsation component on the left side of the drawing appears smaller than a peak of the body motion component on the right side of the drawing. If the maximum amplitude in the sum signal is detected in this case, the body motion component that is not to be measured will be measured.

As illustrated in FIG. 9, however, in the difference signal, the pulsation component is canceled, and only the body motion pulsation remains, as in the aforementioned case.

Thus, as illustrated in FIG. 10, if the differential signal between the sum signal and the difference signal is calculated, the body motion component is canceled, and only the pulsation component remains. Therefore, the detection of the frequency with the maximum amplitude in the differential signal makes it possible to measure the pulsation period of the living body 500.

As explained above, according to the measuring instrument in the first example, it is possible to extract only particular information from various types of information included in the detection signals. It is therefore possible to remove the body motion component, which is unnecessary for the measurement, and to accurately measure the pulsation period of the living body 500

### 2: Second Example

Next, a measuring instrument according to a second example will be explained. The second example has a partially different configuration and operation from those of the first example described above, and is substantially the same in the other points. Therefore, hereinafter, the different point from the first example described above will be explained in details, and an explanation of the same point will be omitted.

<2-1: Configuration of Measuring Instrument>

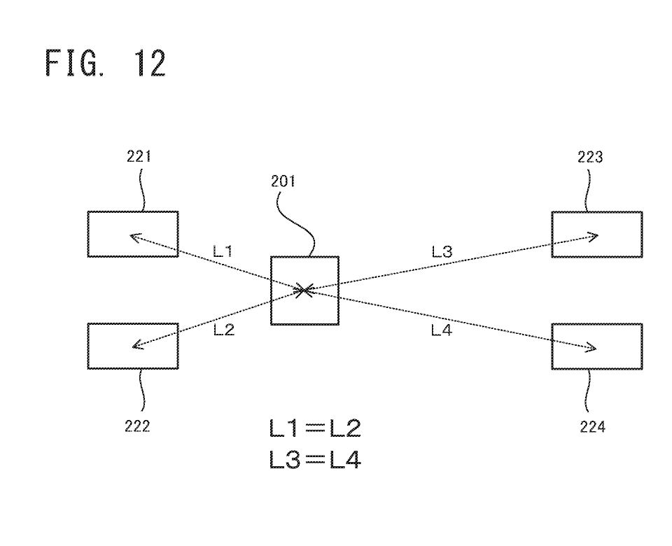

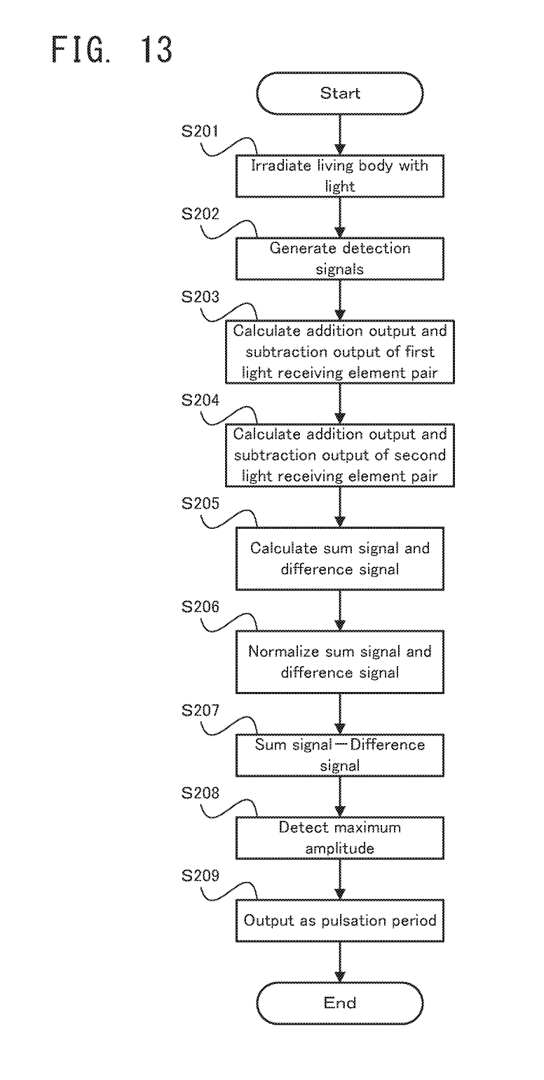

Firstly, a configuration of the measuring instrument according to the second example will be explained with reference to FIG. 11 and FIG. 12. FIG. 11 is a plan view illustrating a distance relation between a light emitting element and light receiving elements of the measuring instrument according to the second example. FIG. 12 is a plan view illustrating a measuring instrument in a modified example according to the second example.

In FIG. 11, the measuring instrument according to the second example is provided with four light receiving elements, which are a first light receiving element 221, a second light receiving element 222, a third light receiving element 223, and a fourth light receiving element 224. Here, a distance L1 between the light emitting point of the light emitting element 201 and a light receiving point of the first light receiving element 221, a distance L2 between the light emitting point of the light emitting element 201 and a light receiving point of the second light receiving element 222, a distance L3 between the light emitting point of the light emitting element 201 and a light receiving point of the third light receiving element 223, and a distance L4 between the light emitting point of the light emitting element 201 and a light receiving point of the fourth light receiving element 224 are set to be equal to each other. Thus, the return light enters the first light receiving element 221, the second light receiving element 222, the third light receiving element 223, and the fourth light receiving element 224 in the same condition.

In FIG. 12, the first light receiving element 221, the second light receiving element 222, the third light receiving element 223, and the fourth light receiving element 224 may be disposed in such a manner that all the distances L1 to L4 are not necessarily equal to each other, but that the distance L1 between the light emitting point of the light emitting element 201 and the light receiving point of the first light receiving element 221 is equal to the distance L2 between the light emitting point of the light emitting element 201 and the light receiving point of the second light receiving element 222, and that the distance L3 between the light emitting point of the light emitting element 201 and the light receiving point of the third light receiving element 223 is equal to the distance L4 between the light emitting point of the light emitting element 201 and the light receiving point of the fourth light receiving element 224.

In other words, the measuring instrument according to the second example may be provided with two light receiving elements pairs, each pair including two light receiving elements having the same distance from the light emitting element 201. Hereinafter, a pair of the first light receiving element 221 and the second light receiving element 222 having the same distance from the light emitting element 201 is referred to as a first light receiving element pair, and a pair of the third light receiving element 223 and the fourth light receiving element 224 is referred to as a second light receiving element pair. Here, the return light enters the two light receiving elements included in the first light receiving element pair, in the same condition. Moreover, the return light also enters the two light receiving elements included in the second light receiving element pair, in the same condition, even though this condition is different from the condition for the first light receiving element pair.

<2-2: Operation of Measuring Instrument>

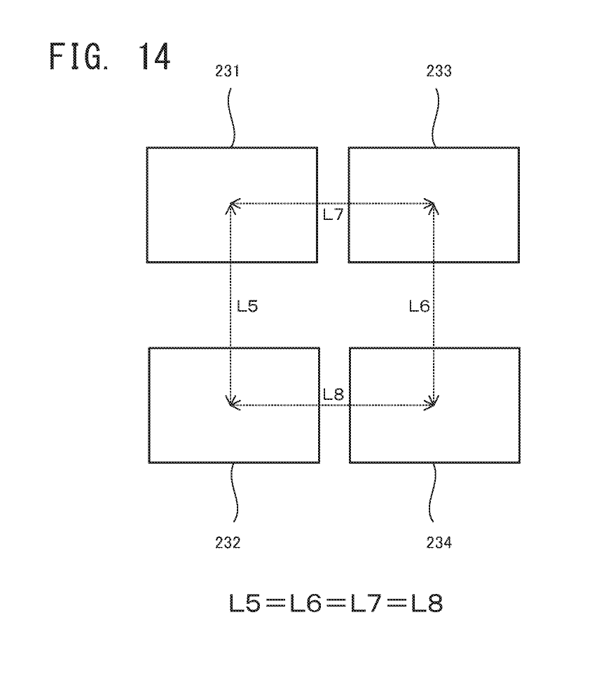

Next, an operation of the measuring instrument according to the second example will be explained with reference to FIG. 13. FIG. 13 is a flowchart illustrating an operation of the measuring instrument according to the second example.

In FIG. 13, in operation of the measuring instrument according to the example, the living body 500 is irradiated with light emitted from the light emitting element 201 (step S201). The light irradiated from the light emitting element 201 is reflected by the living body 500, and is received by each of the first light receiving element 221, the second light receiving element 222, the third light receiving element 223, and the fourth light receiving element 224. Then, on the first light receiving element 221, the second light receiving element 222, the third light receiving element 223, and the fourth light receiving element 224, detection signals are respectively generated according to the intensity of the received light (step S202). In other words, four types of detection signals are separately generated on the first light receiving element 221, the second light receiving element 222, the third light receiving element 223, and the fourth light receiving element 224. The detection signals are outputted to the biological information calculation unit 320.

On the biological information calculation unit 320, firstly, an addition output and a subtraction output of the first light receiving element pair are calculated (step S203). Specifically, the detection signal of the first light receiving element 221 and the detection signal of the second light receiving element 222 are added, to calculate the addition output. Moreover, one of the detection signal of the first light receiving element 221 and the detection signal of the second light receiving element 222 is subtracted from the other, to calculate the subtraction output.

In the same manner, on the biological information calculation unit 320, an addition output and a subtraction output of the second light receiving element pair are calculated (step S204). Specifically, the detection signal of the third light receiving element 223 and the detection signal of the fourth light receiving element 224 are added, to calculate the addition output. Moreover, one of the detection signal of the third light receiving element 223 and the detection signal of the fourth light receiving element 224 is subtracted from the other, to calculate the subtraction output.

Then, on the biological information calculation unit 320, a sum signal and a difference signal are calculated (step S205). Specifically, the addition output of the first light receiving element pair and the addition output of the second light receiving element pair are added, to calculate the sum signal. Moreover, the subtraction output of the first light receiving element pair and the subtraction output of the second light receiving element pair are added, to calculate the difference signal.

Then, on the biological information calculation unit 320, the sum signal and the difference signal are normalized (step S205). Specifically, the sum signal is divided by a maximum value of the sum signal, to be normalized. In the same manner, the difference signal is divided by a maximum value of the difference signal, to be normalized. Another normalization method can be also used.

Then, on the biological information calculation unit 320, the normalized difference signal is subtracted from the normalized sum signal (step S207). In other words, a differential signal between the sum signal and the difference signal is calculated. Then, on the biological information calculation unit 320, a frequency with maximum amplitude is detected in the calculated differential signal (step S208), and the detected frequency is outputted as the pulsation period of the living body (step S209).

Here, in the second example, although the four light receiving elements are provided unlike the first example; the return light enters the two light receiving elements in the same condition in each of the first light receiving element pair and the second light receiving element pair. Thus, the sum signal obtained by adding the respective addition outputs of the first light receiving element pair and the second light receiving element pair includes both the pulsation component and the body motion component, as illustrated in FIG. 5 and FIG. 8. On the other hand, the difference signal obtained by adding the respective subtraction outputs of the first light receiving element pair and the second light receiving element pair includes only the body motion component, as illustrated in FIG. 6 and FIG. 9. Thus, the differential signal between the sum signal and the difference signal includes only the pulsation component, as illustrated in FIG. 7 and FIG. 10.

As explained above, according to the measuring instrument in the second example, it is possible to remove the body motion component, which is unnecessary for the measurement, and to accurately measure the pulsation period of the living body 500. Moreover, particularly in the second example, more light receiving elements are provided in comparison with the first example, and the pulsation component and the body motion component can be accurately extracted by that much.

### 3: Third Example

Next, a measuring instrument according to a third example will be explained. The third example has a partially different configuration and operation from those of the first and second examples described above, and is substantially the same in the other points. Therefore, hereinafter, the different point from the first and second examples described above will be explained in details, and an explanation of the same point will be omitted.

<3-1: Configuration of Measuring Instrument>

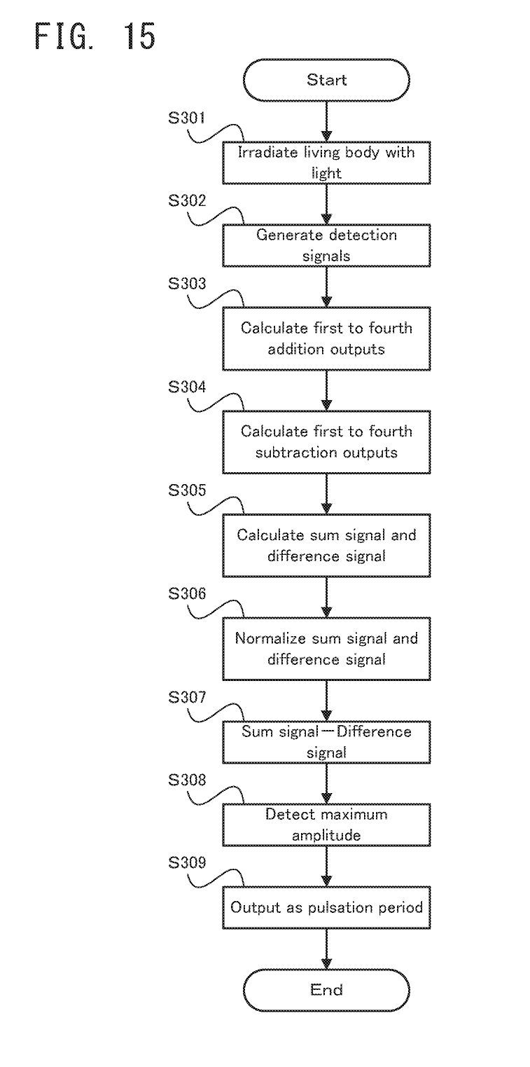

Firstly, a configuration of the measuring instrument according to the third example will be explained with reference to FIG. 14. FIG. 14 is a plan view illustrating a distance relation between a light emitting element and light receiving elements of the measuring instrument according to the third example.

In FIG. 14, the measuring instrument according to the third example is provided with four light receiving elements, which are a first light receiving element 231, a second light receiving element 232, a third light receiving element 233, and a fourth light receiving element 234. Although FIG. 14 does not illustrate the light emitting element 201, the light emitting element 201 is disposed opposite the living body 500, as viewed from the first light receiving element 231, the second light receiving element 232, the third light receiving element 233, and the fourth light receiving element 234. In other words, the measuring instrument according to the third example is a transmission type apparatus. As in the second example, the light emitting element 201 is positioned in such a manner that a distance L1 between the light emitting point of the light emitting element 201 and a light receiving point of the first light receiving element 231, a distance L2 between the light emitting point of the light emitting element 201 and a light receiving point of the second light receiving element 232, a distance L3 between the light emitting point of the light emitting element 201 and a light receiving point of the third light receiving element 233, and a distance L4 between the light emitting point of the light emitting element 201 and a light receiving point of the fourth light receiving element 234 are set to be equal to each other. Thus, the return light enters the first light receiving element 231, the second light receiving element 232, the third light receiving element 233, and the fourth light receiving element 234 in the same condition.

Moreover, particularly in the measuring instrument according to the third example, the first light receiving element 231, the second light receiving element 232, the third light receiving element 233, and the fourth light receiving element 234 are disposed to be adjacent to each other at equal intervals, as viewed in a planar manner. Specifically, the first to fourth light receiving elements are disposed in such a manner that a distance L5 between the light receiving point of the first light receiving element 231 and the light receiving point of the second light receiving element 232, a distance L6 between the light receiving point of the third light receiving element 233 and the light receiving point of the fourth light receiving element 234, a distance L7 between the light receiving point of the first light receiving element 231 and the light receiving point of the third light receiving element 233, and a distance L8 between the light receiving point of the second light receiving element 232 and the light receiving point of the fourth light receiving element 234 are equal to each other.

In the measuring instrument according to the third example, the four light receiving elements are required to be disposed at equal intervals in a relatively narrow space, as described above. Thus, the transmission type apparatus is exemplified in which the light emitting element 201 can be disposed on a different plane from the light receiving elements. If the light emitting element 201 can be disposed at the center of the light receiving elements as in the second example (e.g. refer to FIG. 11), the measuring instrument can be also configured as a reflection type apparatus.

<3-2: Operation of Measuring Instrument>

Next, an operation of the measuring instrument according to the third example will be explained with reference to FIG. 15. FIG. 15 is a flowchart illustrating an operation of the measuring instrument according to the third example.

In FIG. 15, in operation of the measuring instrument according to the example, the living body 500 is irradiated with light emitted from the light emitting element 201 (step S301). The light irradiated from the light emitting element 201 is transmitted through the living body 500, and is received by each of the first light receiving element 231, the second light receiving element 232, the third light receiving element 233, and the fourth light receiving element 234. Then, on the first light receiving element 231, the second light receiving element 232, the third light receiving element 233, and the fourth light receiving element 234, detection signals are respectively generated according to the intensity of the received light (step S302). In other words, four types of detection signals are separately generated on the first light receiving element 231, the second light receiving element 232, the third light receiving element 233, and the fourth light receiving element 234. The detection signals are outputted to the biological information calculation unit 320.

On the biological information calculation unit 320, firstly, an addition output regarding adjacent two light receiving elements of the four light receiving elements is calculated in each adjacent direction (step S303). Specifically, regarding the adjacency in a vertical direction in FIG. 14 (hereinafter referred to as “one direction”, as occasion demands), the detection signal of the first light receiving element 231 and the detection signal of the second light receiving element 232 are added, to calculate a first addition output. Moreover, the detection signal of the third light receiving element 233 and the detection signal of the fourth light receiving element 234 are added, to calculate a second addition output. On the other hand, regarding the adjacency in a horizontal direction in FIG. 14 (hereinafter referred to as “another direction”, as occasion demands), the detection signal of the first light receiving element 231 and the detection signal of the third light receiving element 233 are added, to calculate a third addition output. Moreover, the detection signal of the second light receiving element 232 and the detection signal of the fourth light receiving element 234 are added, to calculate a fourth addition output.

In the same manner, on the biological information calculation unit 320, a subtraction output regarding adjacent two light receiving elements of the four light receiving elements is also calculated in each adjacent direction (step S304). Specifically, regarding the adjacency in the vertical direction in FIG. 14, one of the detection signal of the first light receiving element 231 and the detection signal of the second light receiving element 232 is subtracted from the other, to calculate a first subtraction output. Moreover, one of the detection signal of the third light receiving element 233 and the detection signal of the fourth light receiving element 234 is subtracted from the other, to calculate a second subtraction output. On the other hand, regarding the adjacency in the horizontal direction in FIG. 14, one of the detection signal of the first light receiving element 231 and the detection signal of the third light receiving element 233 is subtracted from the other, to calculate a third subtraction output. Moreover, one of the detection signal of the second light receiving element 232 and the detection signal of the fourth light receiving element 234 is subtracted from the other, to calculate a fourth subtraction output.

Then, on the biological information calculation unit 320, a sum signal and a difference signal are calculated (step S305). Specifically, the first addition output and the second addition output are added, to generate a sum signal regarding one direction. Moreover, the first subtraction output and the second subtraction output are added, to generate a difference signal regarding one direction. In the same manner, the third addition output and the fourth addition output are added, to generate a sum signal regarding another direction. Moreover, the third subtraction output and the fourth subtraction output are added, to generate a difference signal regarding another direction. In other words, the sum signal and the difference signal are generated regarding each of one direction and another direction.

Then, on the biological information calculation unit 320, the sum signal and the difference signal are normalized (step S306). Specifically, the sum signal regarding one direction is divided by a maximum value of the sum signal regarding one direction, to be normalized. The sum signal regarding another direction is divided by a maximum value of the sum signal regarding another direction, to be normalized. In the same manner, the difference signal regarding one direction is divided by a maximum value of the difference signal regarding one direction, to be normalized. The difference signal regarding another direction is divided by a maximum value of the difference signal regarding another direction, to be normalized. Another normalization method can be also used.

Then, on the biological information calculation unit 320, the normalized difference signal is subtracted from the normalized sum signal (step S307). In other words, a difference between the sum signal regarding one direction and the difference signal regarding one direction is calculated, and a difference between the sum signal regarding another direction and the difference signal regarding another direction is calculated. In other words, a differential signal between the sum signal and the difference signal is generated regarding each of one direction and another direction. Then, on the biological information calculation unit 320, a frequency with maximum amplitude is detected in the calculated differential signal (step S308), and the detected frequency is outputted as the pulsation period of the living body (step S309). In the example, since the difference is calculated regarding each of one direction and another direction, the pulsation period of the living body based on the difference regarding one direction is calculated, and the pulsation period of the living body based on the difference regarding another direction is also calculated. The pulsation period calculated in each direction is, for example, selected and outputted. Alternatively, a predetermined arithmetic operation (e.g. an average value calculation, etc.) is performed on the pulsation period of the living body based on the difference regarding one direction and the pulsation period of the living body based on the difference regarding another direction. This also makes it possible to output the pulsation period based on both the difference regarding one direction and the difference regarding another direction.

In the third example, as opposed to the first and second examples, when the first to fourth addition outputs and the first to fourth subtraction outputs are calculated in each adjacent direction regarding the four light receiving elements, the first to fourth addition outputs and the first to fourth subtraction outputs are calculated in the same condition because the four light receiving elements have an equal adjacent distance. Then, the sum signal obtained by adding the first to fourth addition outputs includes both the pulsation component and the body motion component, as illustrated in FIG. 5 and FIG. 8. On the other hand, the difference signal obtained by adding the first to fourth subtraction outputs includes only the body motion component, as illustrated in FIG. 6 and FIG. 9. Thus, the differential signal between includes only the pulsation component, as illustrated in FIG. 7 and FIG. 10.

As explained above, according to the measuring instrument in the third example, it is possible to remove the body motion component, which is unnecessary for the measurement, and to accurately measure the pulsation period of the living body 500. Moreover, particularly in the third example, the addition output and the subtraction output are calculated in each adjacent direction, and thus, the pulsation component and the body motion component can be accurately extracted.

The present invention is not limited to the aforementioned embodiments, but various changes may be made, if desired, without departing from the essence or spirit of the invention which can be read from the claims and the entire specification. A measuring instrument that involves such changes is also intended to be within the technical scope of the present invention.

### DESCRIPTION OF REFERENCE NUMERALS

101 measuring instrument
111 probe
201 light emitting element
211, 221, 231 first light receiving element
212, 222, 232 second light receiving element
223, 233 third light receiving element
224, 234 fourth light receiving element
250 shielding plate
310 wire part
320 biological information calculation unit
330 display
500 living body

1. A measuring instrument comprising:
a light emitting device configured to irradiate light;
a first light receiving device and a second light receiving device configured to receive return light of the irradiated light, which returns from an object to be measured; and
a calculating device configured to calculate information regarding the object to be measured, on the basis of a differential signal which is subtracted a difference signal which is calculated from the light received by the first light receiving device and the light received by the second light receiving device from a sum signal which is calculated from the light received by the first light receiving device and the light received by the second light receiving device.

2. The measuring instrument according to claim 1, wherein a distance between a light emitting point of the light emitting device and the first light receiving device is equal to a distance between the light emitting point of the light emitting device and the second light receiving device.

3. A measuring instrument comprising:
a light emitting device configured to irradiate light;
a first light receiving device pair and a second light receiving device pair, each of which has two light receiving devices configured to receive return light of the irradiated light, which returns from an object to be measured;
a sum signal generating device configured to add an output of one light receiving device and an output of the other light receiving device to make an addition output, in each of the first light receiving device pair and the second light receiving device pair, and configured to add the addition output of the first light receiving device pair and the addition output of the second light receiving device pair to generate a sum signal;
a difference signal generating device configured to subtract one of an output of one light receiving device and an output of the other light receiving device from the other to make a subtraction output, in each of the first light receiving device pair and the second light receiving device pair, and configured to add the subtraction output of the first light receiving device pair and the subtraction output of the second light receiving device pair to generate a difference signal; and
the differential signal which is subtracted the difference signal from the sum signal.

4. The measuring instrument according to claim 3, wherein
a distance between a light emitting point of the light emitting device and one light receiving device of the first light receiving device pair is equal to a distance between the light emitting point of the light emitting device and the other light receiving device of the first light receiving device pair, and
a distance between the light emitting point of the light emitting device and one light receiving device of the second light receiving device pair is equal to a distance between the light emitting point of the light emitting device and the other light receiving device of the second light receiving device pair.

5. A measuring instrument comprising:
a light emitting device configured to irradiate light;
a first light receiving device, a second light receiving device, a third light receiving device, and a fourth light receiving device, configured to receive return light of the irradiated light, which returns from an object to be measured;
a first arithmetic operating device configured to arithmetically operate a first addition output and a first subtraction output, the first addition output being obtained by adding outputs of the first light receiving device and the second light receiving device, which are adjacent to each other in one direction, and the first subtraction output being obtained by subtracting one of the outputs of the first light receiving device and the second light receiving device from the other;
a second arithmetic operating device configured to arithmetically operate a second addition output and a second subtraction output, the second addition output being obtained by adding outputs of the third light receiving device and the fourth light receiving device, which are adjacent to each other in the one direction, and the second subtraction output being obtained by subtracting one of the outputs of the third light receiving device and the fourth light receiving device from the other;
a third arithmetic operating device configured to arithmetically operate a third addition output and a third subtraction output, the third addition output being obtained by adding outputs of the first light receiving device and the third light receiving device, which are adjacent to each other in another direction that is different from the one direction, and the third subtraction output being obtained by subtracting one of the outputs of the first light receiving device and the third light receiving device from the other;
a fourth arithmetic operating device configured to arithmetically operate a fourth addition output and a fourth subtraction output, the fourth addition output being obtained by adding outputs of the second light receiving device and the fourth light receiving device, which are adjacent to each other in the another direction, and the fourth subtraction output being obtained by subtracting one of the outputs of the second light receiving device and the fourth light receiving device from the other;
a first sum signal generating device configured to add the first addition output and the second addition output to generate a sum signal regarding the one direction;
a first difference signal generating device configured to add the first subtraction output and the second subtraction output to generate a difference signal regarding the one direction;
a second sum signal generating device configured to add the third addition output and the fourth addition output to generate a sum signal regarding the another direction;
a second difference signal generating device configured to add the third subtraction output and the fourth subtraction output to generate a difference signal regarding the another direction; and
the differential signal which is subtracted the difference signal from the sum signal, in each of the one direction and the another direction.

6. The measuring instrument according to claim 5, wherein the first light receiving device, the second light receiving device, the third light receiving device, and the fourth light receiving device are positioned to have an equal adjacent distance in a planar manner.

7. The measuring instrument according to claim 5, wherein the first light receiving device, the second light receiving device, the third light receiving device, and the fourth light receiving device have an equal distance from a light emitting point of the light emitting device.

8. The measuring instrument according to claim 1, wherein the calculating device calculates the information regarding the object to be measured, on the basis of the differential signal indicating a difference frequency component which is subtracted a frequency component of the difference signal from a frequency component of the sum signal.

9. The measuring instrument according to claim 8, wherein
the measuring instrument further comprises a normalizing device configured to divide the frequency component of the sum signal and the frequency component of the difference signal by respective maximum values, to be normalized, and
the calculating device calculates the information regarding the object to be measured, on the basis of the differential signal indicating a difference frequency component, which is subtracted the normalized frequency component of the difference signal from the normalized frequency component of the sum signal.

10. The measuring instrument according to claim 9, wherein the calculating device estimates a frequency that indicates a maximum amplitude in the difference frequency component, to be a pulsation period of the object to be measured.

11. The measuring instrument according to claim 6, wherein the first light receiving device, the second light receiving device, the third light receiving device, and the fourth light receiving device have an equal distance from a light emitting point of the light emitting device.

12. The measuring instrument according to claim 3, wherein the calculating device calculates the information regarding the object to be measured, on the basis of the differential signal indicating a difference frequency component which is subtracted a frequency component of the difference signal from a frequency component of the sum signal.

13. The measuring instrument according to claim 5, wherein the calculating device calculates the information regarding the object to be measured, on the basis of the differential signal indicating a difference frequency component which is subtracted a frequency component of the difference signal from a frequency component of the sum signal.
